(19) United States
(12) Patent Application Publication
(10) Pub. No.: US 2026/0232171 A1
(43) Pub. Date: Aug. 13, 2026

(54) Method of Using a Single Bus, USB Signal Transfer Endoscope System

(71) Applicant: Roland Quaye, Fredericksburg, VA (US)

(72) Inventor: Roland Quaye, Fredericksburg, VA (US)

(21) Appl. No.: 19/053,164

(22) Filed: Feb. 13, 2025

(51) Int. Cl. A61B 1/00

(52) U.S. Cl. CPC A61B 1/00011; A61B 1/00042; A61B 1/00114

(57) ABSTRACT

The present invention generally relates to a USB signal transfer endoscope and its method of use. The present invention is a method that utilizes an endoscope system with a detachable device that provides power to the specialty electronics located in the control handle of the endoscope. The specialty electronics eliminates the need for external processing units. Less bulky equipment improves the user experience because any computer or phone with the application (App.) can receive images, e.g., photos and videos, for end users to view worldwide. The portability and ease of use make the unit less costly and enable the creation of single-use endoscopes. Single endoscopes reduce or eliminate cross-contamination and reprocessing issues.

### BACKGROUND

This patent disclosure generally relates to endoscopes. An endoscope is a medical instrument used to visually examine internal body cavities and organs. It consists of a long, flexible tube with a light and camera at the end, which transmits images to a monitor for a medical professional to observe and monitor. Moreover, it relates to the transfer of information via specialty electronics for processing and the interface with a computer to execute the commands to capture images.

A traditional endoscope uses an additional processing unit to process images and execute several commands such as image storage, print, record annotations on images, etc. The traditional method comes at a high cost and makes the overall cost of endoscopes very high. For example, a traditional imaging endoscope includes a control handle and a processor in a control cabinet. The endoscope is communicatively coupled and controlled coupled to the control handle. The control cabinet is communicatively coupled to the control handle and functions to provide image processing capabilities and a power supply for the endoscope. The control cabinet is typically mounted on wheels so that it can easily be placed near a patient prior to the procedure. The control cabinet is connected to a source of electrical power, either AC mains or a battery. The control cabinet includes a suite of application software. Application software includes a graphical user interface (GUI) software application, a system control software application, and a network software application. In addition, the control cabinet has a series of system electronics, an imaging electronics board, and a network connection. The former system is bulky and expensive. Thus, there exists a need for endoscopes that don't require additional processors that can interface with computer software that enables all the functions of image storage, image capture, image freeze, print, and record annotations.

### SUMMARY

The present invention generally relates to a USB signal transfer endoscope and its method of use. The present invention is a method that utilizes an endoscope system with a detachable device that provides power to the specialty electronics located in the control handle of the endoscope. The specialty electronics eliminates the need for external processing units. Less bulky equipment improves the user experience because any computer or phone with the application (App.) can receive images, e.g., photos and videos, for end users to view worldwide. The portability and ease of use make the unit less costly and enable the creation of single-use endoscopes. Single endoscopes reduce or eliminate cross-contamination and reprocessing issues.

### BRIEF DESCRIPTION OF THE DRAWINGS

FIG. 1 is a perspective of an endoscope.

FIG. 2 is a close-up view of the control section.

FIG. 3 is a cut-away view of the control handle.

FIG. 4 shows the USB connection means.

FIG. 5 is a flow diagram depicting the information flow between the components.

FIG. 6 is a block diagram of the custom electronics.

### DETAILED DESCRIPTION

The disclosed signal transfer endoscope system will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of signal transfer endoscopes are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated. “Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

### Method of Using a Single Bus, USB Signal Transfer Endoscope System

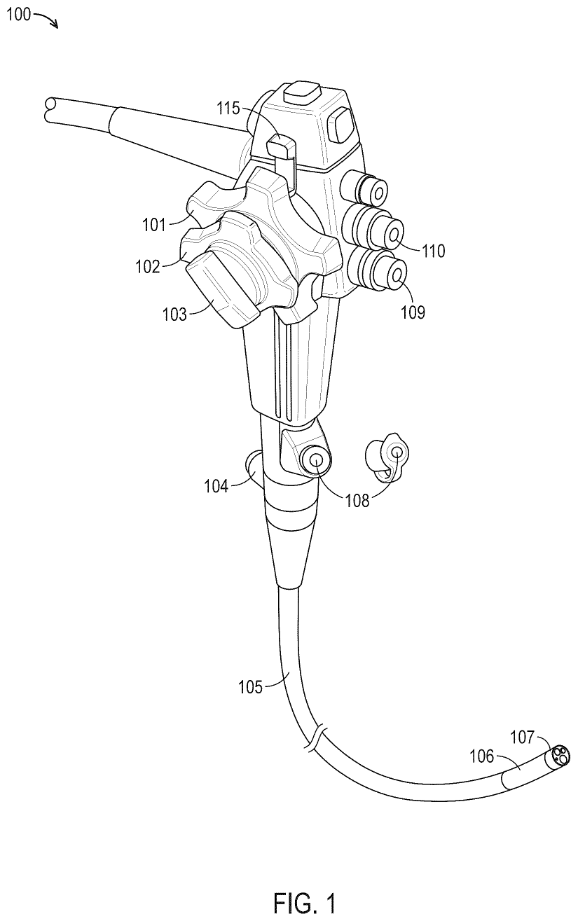

With reference to FIGS. 1-6, the METHOD OF USING A SINGLE BUS, USB SIGNAL TRANSFER ENDOSCOPE SYSTEM will now be described. FIG. 1 displays the parts of an endoscope, 100. An endoscope comprises a control section having an up/down control knob, 101; an up/down angulation lock, 115; a right/left angulation control knob, 102; and a right/left angulation lock, 103. The endoscope also includes an auxiliary water inlet, 104; a flexible portion, 105; a bendable portion, 106; and the distal end, 107. The endoscope further includes a channel opening, 108; an air/water valve, 109; a suction valve, 110. The distal end of the endoscope can comprise a plurality of devices with non-limiting examples, including lights, an objective lens, an air/water nozzle, an instrument channel, or any device necessary for the procedure.

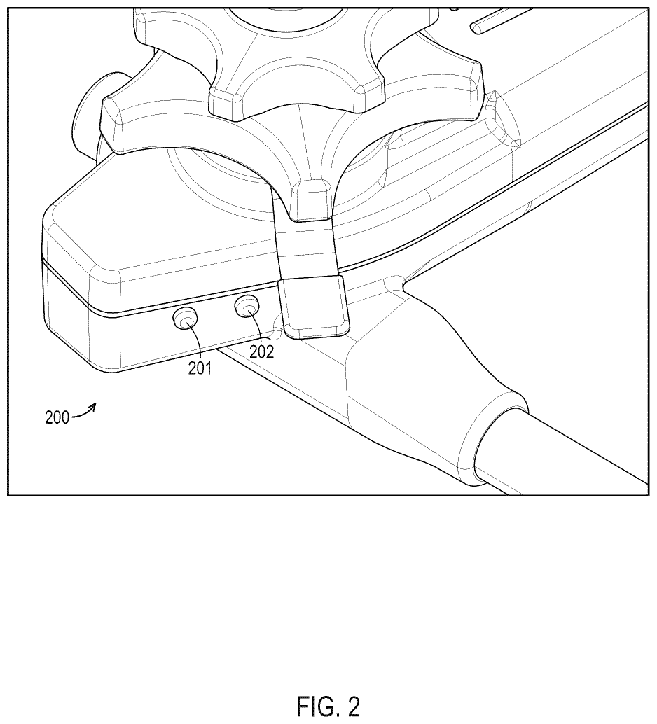

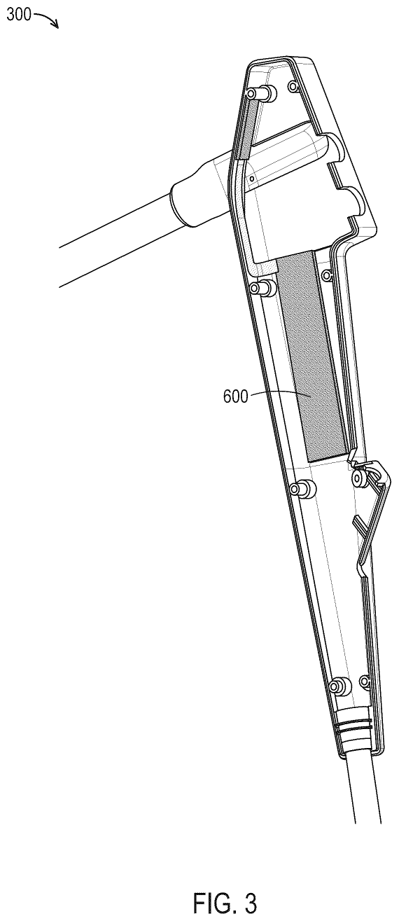

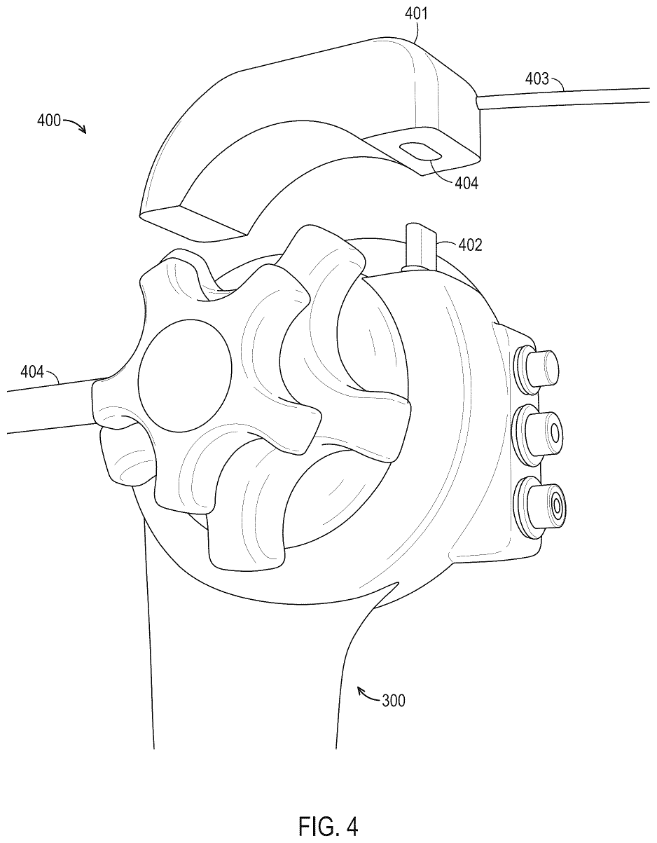

FIG. 2 shows the specialty buttons 200 that freeze the image, 202 and capture the image, 201. FIG. 3 shows a cut-away portion of the control handle 300 and the specialty electronics 600. FIG. 4 shows the connection means 400 to connect the endoscope to a control means via a detachable connection means 401, which communicatively couples with the control handle, 300, having a male USB port 402, via the female USB port 404, and a cable 403 that couples to the control means. The control means is an App. installed on any device that can execute the application software, with non-limiting examples such as a server, desktop or laptop computer, a phone, or a tablet. The system may be communicatively coupled to peripherals, with non-limiting examples including a visual display, such as a monitor or screen, a printer, or a modem.

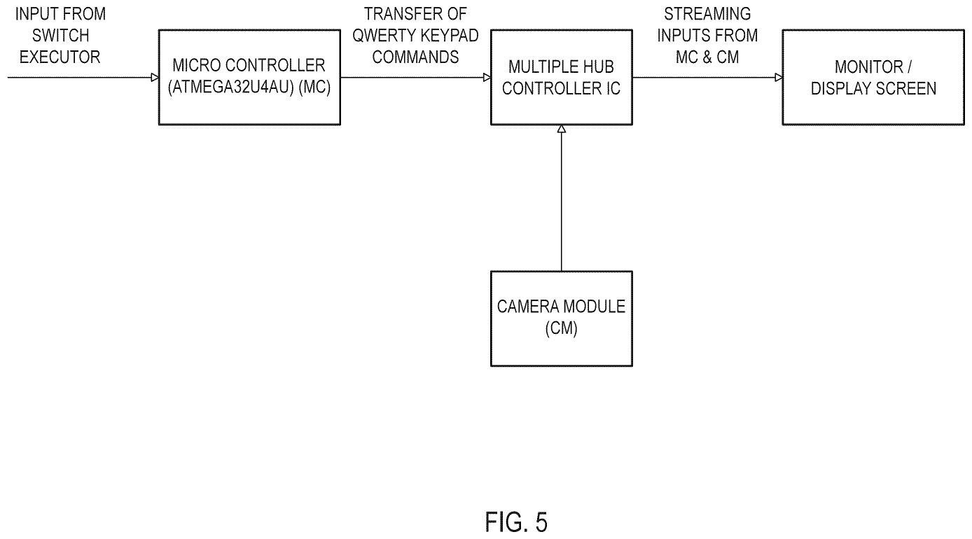

FIG. 5 is a flow diagram showing the camera module taking in an image signal that is multiplexed on the circuit board 600. The multiplexed image signal is then taken to the computer from the circuit board 600, thereby allowing for image control and video streaming.

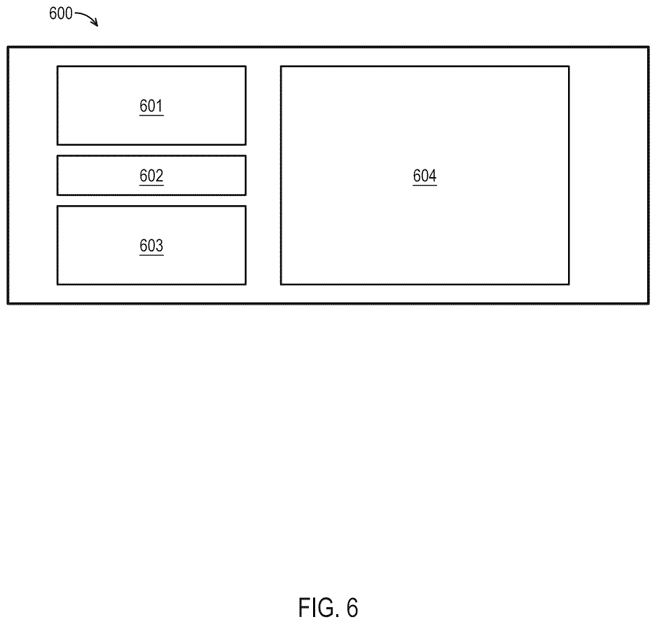

FIG. 6 is a block diagram of the specialty electronics, which comprises a circuit board 600, an ISP programmer 601, a power supply filter circuit 602, a USB 2.0 high-speed controller 603, and a microcontroller 604.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions, and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

1. A USB signal transfer endoscope system comprising:
a control handle having at least one control knob, at least one angulation lock, and at least one button disposed on the control handle; a cylindrical section coupled to the control handle; where the cylindrical section further comprises a flexible portion and a bendable portion; where the bendable portion has an end distal to the control handle; where disposed on the distal end of the bendable portion are a plurality of devices; where disposed on the control handle's end, opposite from the distal end of the bendable portion, is a means to connect to a USB interface.

2. The USB signal transfer endoscope system of claim 1 includes a specialty electronics where the specialty electronics is disposed within the control handle; where the specialty electronics further a comprises a circuit board, an ISP programmer, a power supply filter circuit, a high-speed USB controller, and a microcontroller, where the specialty electronics is communicatively coupled via the USB interface to an App; where the App is installed on a device.

3. The USB signal transfer endoscope system of claim 2 where the App. is communicatively coupled to at least one peripheral.

4. A method for USB signal transfer comprising: the USB signal transfer endoscope system of claim 3 where a camera module acquires an image signal, which is then multiplexed on the circuit board; then the multiplexed image signal is sent from the circuit board to the device.

5. Where the image signal of claim 4 is acquired by pressing the button on the control handle.

6. Where the image signal of claim 4 is a picture.

7. Where the image signal of claim 4 is a video.

8. The method of claim 4 where executing a computer code command acquires the image signal without using the button.

9. The method of claim 4 where translating the image signal into a computer code creates a video or a picture.
